(19) United States
(12) Patent Application Publication
(10) Pub. No.: US 2010/0062025 A1
(43) Pub. Date: Mar. 11, 2010

(54) Use of Clostridium perfringens type C bacterium for the manufacture of a vaccine

(75) Inventors: Maarten Hendrik Witvliet, Boxmeer (NL); Jozef Franciscus Maria Smeets, Boxmeer (NL); Keith Redhead, Milton Keynes (GB)

Correspondence Address: Intervet/Schering-Plough Animal Health, Patent Dept. K-6-1, 1990, 2000 Galloping Hill Road, Kenilworth, NJ, 07033-0530, US

(73) Assignee: Intervet International B.V., Boxmeer (NL)

(21) Appl. No.: 12/514,830

(22) PCT Filed: Nov. 19, 2007
(86) PCT No.: PCT/EP2007/062484

§ 371 Date: May 14, 2009

Related U.S. Application Data
(60) Provisional application No. 60/860,447, filed on Nov. 20, 2006.

(30) Foreign Application Priority Data
Nov. 20, 2006 (EP) 06124404.2

(51) Int. Cl. A61K 39/08; A61P 37/04

(52) U.S. Cl. 424/247.1

(57) ABSTRACT

The present invention relates to the use of a vaccine comprising Clostridium perfringens type C bacterium for protecting swine against Clostridium perfringens type A infection.

The present invention relates to the use of Clostridium perfringens type C bacterium for the manufacture of a vaccine.

Clostridia are Gram-positive spore-forming anaerobic bacteria. The Clostridia are widely recognized as pathogens of both domestic and wild animals. An overview of the various Clostridia and the animal diseases they cause is given by Songer, J. G. in The Clostridia; Chapter 10: Clostridial Diseases of Animals, Molecular biology and Pathogenesis, ed. Rood, J. I. et al., Academic Press Ltd (1997) ISBN 0-12-595020-9.

Of the currently known Clostridial species, Clostridium perfringens is sometimes considered to be the most widely occurring bacterial pathogen, and of all Clostridial species it certainly is the most important cause of Clostridial enteric disease in domestic animals.

The species Clostridium perfringens is sub-divided in five types; type A-E, on the basis of the major toxins they make.

Clostridium perfringens types A and C, and Clostridium difficile are the principal enteric clostridial pathogens of swine.

Clostridial enteric infections in pigs have more specifically been described by Songer, J. G. and Uzal, F. A. (J. Vet. Diagn. Invest 17: 528-536 (2005)).

Clostridium perfringens type C infects pigs, cattle, chickens, humans, sheep, dogs and horses. Piglets are the most vulnerable to Clostridium perfringens type C infection. In piglets 1-2 days old, the infection causes morbidity rates of between 30%-50% and case fatality rates of 50%-100%. Older piglets (1-2 weeks old) may have a longer course of infection.

Sows are the common source of infection.

The common approach for the protection of piglets is vaccination of sows with a Clostridium perfringens type C vaccine, in order to induce antibodies that are transferred to their offspring via colostrum. This leads to a high level of protection in piglets at that moment in time at which they are the most vulnerable to Clostridial infection. Such vaccines are based upon toxoids. Usually, they are detoxified supernatant vaccines, in which no cells are present. Toxoid vaccines have been described i.a. by Springer, S, and Selbitz, H.-J. (FEMS Immun. and Medical Microbiol. 24: 333-336 (1999)).

Clostridium perfringens type A infects lambs, goats, calves, chickens, pigs horses, dogs and humans. In fully developed pigs, Clostridium perfringens type A is a member of the normal flora of the intestine. It is therefore currently assumed that fully developed pigs are not adversely affected by the presence of Clostridium perfringens type A in the intestine.

It can however cause porcine necrotic enterocolitis in neonatal and weaned pigs. Vaccines for the protection of pigs against Clostridium perfringens type A are rare. There is currently only one (conditionally licensed) vaccine commercially available.

One of the reasons for this may be the fact that, as said above, Clostridium perfringens type A is a member of the normal flora. Therefore, one would not expect it to trigger the immune system. As a consequence, one would not expect antibody-production to be induced against Clostridium perfringens type A. If antibodies were induced, Clostridium perfringens type A would not survive in the gut, let alone be a member of the normal flora. As a further consequence, one would of course not expect sows to transmit any protection to their offspring through the colostrum. For whatever reason, it seems that Clostridium perfringens type A does not induce sufficient immune stimulation.

It has now been surprisingly found, that Clostridium perfringens type C vaccines comprising detoxified culture supernatant are very capable of inducing cross-protection against Clostridium perfringens type A infection.

More surprising, it was found that vaccination of sows with Clostridium perfringens type C culture supernatant leads to a significant protection of their piglets against Clostridium perfringens type A infection.

Even more surprising, it was found that even the sows perform better after this vaccination. This is surprising, because it was generally assumed, as mentioned above, that fully developed pigs, such as sows are not adversely affected by Clostridium perfringens type A infection. This finding for the first time proves the opposite to be the case.

Therefore, a first embodiment of the present invention relates to the use of a Clostridium perfringens type C bacterium for the manufacture of a vaccine for the protection of fully developed pigs against Clostridium perfringens type A infection.

A second embodiment of the present invention relates to the use of a Clostridium perfringens type C bacterium for the manufacture of a vaccine for the protection of sows against Clostridium perfringens type A infection.

A third embodiment of the present invention relates to the use of a Clostridium perfringens type C bacterium for the manufacture of a vaccine for sows, for the protection of piglets against Clostridium perfringens type A infection.

A fourth embodiment of the present invention relates to the use of a Clostridium perfringens type C bacterium for the manufacture of a vaccine for the protection of piglets against Clostridium perfringens type A infection.

As the skilled artisan will understand, in fact the use of a Clostridium perfringens type C bacterium for the manufacture of a vaccine is a two step process: the Clostridium perfringens type C bacterium is used for growth in a culture medium, in order to obtain the culture supernatant that is the basis for the manufacture of the vaccine according to the invention.

Therefore, another embodiment of the present invention relates to the use of a Clostridium perfringens type C culture supernatant for the manufacture of a vaccine for the protection of fully developed pigs against Clostridium perfringens type A infection.

Still another embodiment of the present invention relates to the use of a Clostridium perfringens type C culture supernatant for the manufacture of a vaccine for the protection of sows against Clostridium perfringens type A infection.

Again another embodiment of the present invention relates to the use of a Clostridium perfringens type C culture supernatant for the manufacture of a vaccine for sows, for the protection of piglets against Clostridium perfringens type A infection.

Also an embodiment of the present invention relates to the use of a Clostridium perfringens type C culture supernatant for the manufacture of a vaccine for the protection of piglets against Clostridium perfringens type A infection.

Within the Clostridium perfringens type C toxin type, a division can be made between those strains that produce beta-2 toxin and those strains that don't. About 40% of all Clostridium perfringens type C strains produce this toxin.

It was found that a Clostridium perfringens type C bacterium that produces beta-2 toxin is even more suitable than a non-beta-2 toxin producing Clostridium perfringens type C bacterium. Such strains and their identification are i.a. described by Fisher, D. J. et al. (Inf. & Immun. 74: 5200-5210 (2006)).

Thus, a preferred form of this embodiment relates to a use wherein a Clostridium perfringens type C bacterium is used that produces beta-2 toxin.

Vaccines manufactured as described above are by no means the only vaccines that are administered to pigs in the process of commercial pig rearing. Clostridial vaccines are frequently given around the same time as vaccines against other pig pathogenic organisms and viruses.

Thus, in another preferred embodiment, the vaccine manufactured as described in the present invention additionally comprises one or more antigens derived from pig pathogenic organisms or viruses, antibodies against those antigens or genetic information encoding such antigens.

Such organisms or viruses are preferably selected from the group of Pseudorabies virus, PRRS virus, Porcine influenza virus, Porcine parvo virus, Transmissible gastro-enteritis virus, Rotavirus, Escherichia coli, Erysipelothrix rhusiopathiae, Bordetella bronchiseptica, Salmonella typhimurium, Salmonella choleraesuis, Haemophilus parasuis, Pasteurella multocida, Streptococcus suis, Mycoplasma hyopneumoniae, Brachyspira hyodysenteriae and Actinobacillus pleuropneumoniae.

Therefore, a more preferred form of this embodiment relates to a vaccine manufactured as described in the present invention that additionally comprises one or more antigens derived from pig pathogenic organisms or viruses, wherein these organisms or viruses are selected from the group of Pseudorabies virus, PRRS virus, Porcine influenza virus, Porcine parvo virus, Transmissible gastro-enteritis virus, Rotavirus, Escherichia coli, Erysipelothrix rhusiopathiae, Bordetella bronchiseptica, Salmonella typhimurium, Salmonella choleraesuis, Haemophilus parasuis, Pasteurella multocida, Streptococcus suis, Mycoplasma hyopneumoniae, Brachyspira hyodysenteriae and Actinobacillus pleuropneumoniae.

Next to Clostridium perfringens infection, piglets frequently suffer from Escherichia coli infection. Escherichia coli is the cause of another severe enteric disease in piglets. Therefore, piglets or pregnant sows are frequently vaccinated with Escherichia coli vaccines. E. coli vaccines are commonly used in the field and they are commercially available.

A combination vaccine comprising both Clostridium perfringens type C culture supernatant and Escherichia coli vaccine components therefore is highly desirable, because it protects against Clostridium perfringens type C, Clostridium perfringens type A and Escherichia coli infection.

Therefore, a most preferred form of this embodiment relates to a vaccine manufactured as described in the present invention that additionally comprises one or more antigens derived from the pig pathogenic organism Escherichia coli.

The invention is exemplified in a non-limiting way in the following examples.

### EXAMPLES

### Production of Clostridium perfringens Type C Antigen

A standard Clostridium perfringens type C strain, strain 587 (also named CWC 1/S, from the Weybridge U.K. Collection) was cultured in a fermentor until the beginning of the stationary growth phase. The culture was centrifuged and the pellet containing the bacterial cells was discarded. Formaldehyde was added to the supernatant to a final concentration of 0.5% v/v to inactivate toxins, followed by concentration by means of ultra filtration and 0.2 μm filtration.

### Vaccine Preparation

A vaccine was formulated by mixing the Clostridium perfringens type C antigen and a tocopherol-based adjuvant. Each dose of vaccine (2 ml) comprised 20 μl of a 17-fold concentrated Clostridium perfringens type C antigen toxoid culture supernatant concentrate.

The vaccine complied with the European Pharmacopoeia monograph 0363 (Clostridium perfringens vaccine for veterinary use) regarding safety and potency of type C vaccines.

Additionally, in this experiment purified E. coli antigens (F4ab, F4ac, F5 and F6 fimbriae and heat-labile toxin) were added to suppress possible E. coli infection. These components are known in the art, and the skilled artisan would know how to make a vaccine on the basis of these components.

Alternatively, however the skilled artisan could use a commercially available and ready-to-use E. coli vaccine, such as Porcilis Porcoli DF (obtainable through Intervet Int. B.V., Wim de Korverstraat 35, Boxmeer, The Netherlands).

The control vaccine comprises the same components except for the Clostridium perfringens type C antigen, that was not present in the control group.

### Protection of Sows.

A Dutch pig production farm on which there was a clinical Clostridium perfringens type A outbreak was selected for a randomized double blinded field efficacy study. The Clostridium perfringens type A isolate from the farm was found to produce both α-toxin and 132-toxin.

Sows were vaccinated IM during pregnancy at approximately 6 and 2 weeks before farrowing with either a vaccine containing E. coli antigens only (control group) or with a vaccine containing E. coli antigens and Clostridium perfringens type C antigen (test group). The vaccines were color-coded (2× test vaccine, 1× control vaccine) to assure blinding, and distribution of the sows over the groups was approximately 2:1 (Table 1).

TABLE 1
Group sizes

| | | Number of live born | Total number of |
|---|---|---|---|
| Group | Number of sows | piglets per sow | piglets |
| Control | 41 | 11.6 | 453 |
| Test | 77 | 12.1 | 904 |

From the day of first vaccination until the time of weaning of the piglets (3-4 weeks after farrowing), the sows were observed every 3 days for general health, feed intake and diarrhea. To facilitate statistical analysis, a clinical scoring system was used (Table 2). Piglets born to the vaccinated sows were observed daily during the first week of life, once every three days from one week until weaning and weekly thereafter up to the end of the nursery period. General health, feed intake, diarrhea and mortality were recorded (Table 2).

TABLE 2
Scoring system used for sows and piglets:

| Clinical | | | Faecal | Faecal |
|---|---|---|---|---|
| Score | Demeanour | Appetite | Consistency | Composition |
| 0 | Normal | Normal | Normal | Normal |
| 1 | Depressed | Slight | Soft | Mucus present |
| | | inappetance | | |
| 2 | Lethargic | Marked | Liquid | Mucus with blood |
| | | inappetance | | present |
| 3 | Moribund | Not suckling | Watery | Blood present with |
| | | | | intestinal casts |

### Results

TABLE 3
Clinical observations of sows

| | | Score (N) | | | | |
|---|---|---|---|---|---|---|
| | | 0 | 1 | 2 | 3 | Mean |
| General | Control | 687 | 99 | 51 | 20 | 0.30 |
| Demeanour | Test | 1623 | 33 | 0 | 0 | 0.02 |
| Appetite | Control | 597 | 139 | 71 | 49 | 0.50 |
| | Test | 1598 | 47 | 9 | 0 | 0.04 |
| Faecal | Control | 385 | 250 | 153 | 68 | 0.89 |
| Consistency | Test | 1564 | 67 | 17 | 7 | 0.07 |
| Faecal | Control | 780 | 71 | 4 | 0 | 0.09 |
| Composition | Test | 1647 | 8 | 0 | 0 | 0.01 |

Table 3 shows the effect of vaccination with a vaccine comprising the Clostridium perfringens type C antigens compared to vaccination with a vaccine without Clostridium perfringens type C antigens.

As follows unambiguously from Table 3, vaccination with the vaccine comprising the Clostridium perfringens type C antigens resulted in a very significant improvement of the health status of the sows.

### Protection of the Offspring of Vaccinated Sows.

Also, the mortality rate in the liters of the sows that had been vaccinated with the vaccine comprising the Clostridium perfringens type C antigens was compared to that in the liters of sows that had been vaccinated with a vaccine without Clostridium perfringens type C antigens.

As follows unambiguously from Table 4, the mortality rate in the liters of the sows that had been vaccinated with the vaccine comprising the Clostridium perfringens type C antigens was significantly lower than in the control group.

In addition, Table 5 shows that piglets from sows that had been vaccinated with the vaccine comprising the Clostridium perfringens type C antigens had significantly lower clinical scores than the control group.

TABLE 4
Weekly mortality in control and test litters

| Age (days) | Control | Test |
|---|---|---|
| 0-6 | 29 | 22 |
| 7-13 | 3 | 3 |
| 14-21 | 7 | 2 |
| 21-27 | 6 | 1 |
| 28-34 | 2 | 1 |
| 35-41 | 0 | 0 |
| 42-48 | 1 | 1 |
| 49-55 | 1 | 1 |
| 56-62 | 1 | 0 |
| 63-70 | 0 | 0 |
| Total (%) | 50 (11.04) | 31 (3.43) |

TABLE 5
Clinical observations of piglets

| | | Score (N) | | | | |
|---|---|---|---|---|---|---|
| | | 0 | 1 | 2 | 3 | Mean |
| General | Control | 336 | 232 | 137 | 73 | 0.93 |
| Demeanour | Test | 1159 | 271 | 55 | 11 | 0.28 |
| Appetite | Control | 426 | 166 | 128 | 58 | 0.77 |
| | Test | 1336 | 125 | 31 | 4 | 0.13 |
| Faecal | Control | 544 | 118 | 71 | 40 | 0.49 |
| Consistency | Test | 1419 | 48 | 17 | 12 | 0.08 |
| Faecal | Control | 636 | 104 | 31 | 6 | 0.24 |
| Composition | Test | 1414 | 70 | 10 | 1 | 0.06 |

### CONCLUSION

the data show that vaccination of sows with a vaccine containing Clostridium perfringens type C antigens results in a significant improvement of the health status of both vaccinated sows and their offspring in face of a Clostridium perfringens type A outbreak.

1-12. (canceled)

13. A method for protecting swine against Clostridium perfringens type A infection, comprising administering a vaccine comprising an immunogenically effective amount of Clostridium perfringens type C antigen.

14. The method of claim 13, wherein the Clostridium perfringens type C antigen is detoxified Clostridium perfringens type C culture supernatant.

15. The method of claim 13, wherein the swine is a sow.

16. A method for protecting piglets against Clostridium perfringens type A infection, comprising administering an immunogenically effective amount of Clostridium perfringens antigen to the mother of the piglets.

17. The method of claim 14, wherein the swine is a sow.

18. The method of claim 17, wherein the sow is a pregnant sow.

19. The method of claim 16, wherein the Clostridium perfringens type C antigen is detoxified Clostridium perfringens type C culture supernatant.

20. The method of claim 13, wherein the Clostridium perfringens type C antigen is from a Clostridium perfringens type C bacterium that produces beta-2 toxin.

21. The method of claim 13, wherein the Clostridium perfringens type C antigen is a Clostridium perfringens type C bacterium.

22. The method of claim 13, wherein the Clostridium perfringens type C antigen is a Clostridium perfringens type C bacterin.

23. The method of claim 13, wherein the vaccine comprises an Escherichia coli antigen.
